(19) United States
(12) Patent Application Publication
(10) Pub. No.: US 2021/0333286 A1
(43) Pub. Date: Oct. 28, 2021

(54) ACTIVITY SENSOR CONTROLS

(71) Applicant: GLYMPSE BIO, INC., Cambridge, MA (US)

(72) Inventors: James Bowen, Cambridge, MA (US); Faycal Touti, Cambridge, MA (US)

(21) Appl. No.: 17/238,599

(22) Filed: Apr. 23, 2021

Related U.S. Application Data
(60) Provisional application No. 63/015,341, filed on Apr. 24, 2020.

(51) Int. Cl. G01N 33/68; G01N 33/574

(52) U.S. Cl. CPC G01N 33/6803; G01N 2333/96494; G01N 33/57492

(57) ABSTRACT

An activity sensor sensitive to enzymes indicative of tissue condition are co-administered with control or normalizing activity sensors providing levels of the same enzyme in other tissues or levels of control enzymes indicative of assay success. Levels of control reporters can be used to normalize activity sensor data across samples such as in analyte velocity analyses. Control reporters can also be used to differentiate localized enzyme activity from systemic activity and to confirm activity sensor localization or to troubleshoot activity sensor uptake problems. Activity sensors and controls sensitive to immunological enzymes are particularly useful in assessing immuno-oncology treatments.

### TECHNICAL FIELD

The invention relates to enzyme activity sensor controls.

### BACKGROUND

Diagnosing and monitoring diseases such as cancer often involves invasive and painful procedures such as tissue biopsies. Biomarkers can provide a non-invasive avenue for tracking internal phenomenon but a limited in their scope by the requirement of a naturally occurring phenomenon-linked molecule and our understanding of that link. Discovering new natural biomarkers is a troubled process. Recent developments include synthetic biomarkers that are engineered to release detectable molecules in response to various conditions in the body. However, confirming the source of reporter data, troubleshooting target-specific reporters, and eliminating variability across samples all present obstacles to accurate reporting using synthetic biomarkers.

Such biomarkers have promising applications in the field of cancer immunotherapy or immuno-oncology (I-O). Immuno-oncology is a recently developed field that has shown promise in treating various forms of cancer. I-O refers to the use of a patient's own immune system to attack their cancer. I-O is a broad category and includes passive as well as active techniques. Passive techniques involve the augmentation of a patient's existing anti-tumor immune response through, for example, immune checkpoint inhibitors (e.g., CTLA-4 blockade, PD-1 inhibitors, or PD-L1 inhibitors) that can disrupt tumor defenses against immune system attacks. Active techniques include targeted immune therapies such as engineered CAR-T cells programmed to target tumor-specific antigens. Unfortunately, gaining insight into the tumor environment without invasive biopsies or potentially misleading imaging is difficult, hence the advantageous application of synthetic biomarkers. That said, it can be difficult to eliminate false negatives in existing non-invasive diagnostic and monitoring assays. Additionally, in the case of cancer immunotherapies, there may be a need to differentiate between localized, anti-tumor immune responses and general immune system activity which can prove difficult, even with information from synthetic biomarkers.

### SUMMARY

The invention provides reporters useful in non-invasive detection and monitoring of disease and therapeutic efficacy. According to the invention, activity sensors are used to validate results, and calibrate diagnostic and therapeutic assays. In one embodiment, the invention comprises the use of normalizer and control reporters for the detection of enzyme activity characteristic of cancer progression, localized immune system activity, and immuno-therapeutic response. Activity sensors, as described herein, provide synthetic biomarkers that can be administered to a patient and localized to a target tissue to release detectable reporter molecules indicative of enzyme activity at the target. Such activity sensors targeted to tumor tissue and engineered to report on enzyme activity indicative of immune response or tumor progression are useful in assessing patient response to immuno-oncological therapies and monitoring disease status. However, analysis of reporter levels in I-O therapies is often comparative and, therefore, relies on an understanding of baseline enzyme activity and requires monitoring of changes in that activity over time. Measurements from samples taken across various time points involve sample-to-sample variability that must be accounted for. Values from control reporters of the invention, acting as normalizers, can help eliminate that variability to provide a more accurate assessment of disease progression and therapeutic response. Additionally, immune system activity is not necessarily anti-tumor activity and, accordingly, assessment of I-O therapies requires differentiation of anti-tumor and general immune responses. Control and normalizer reporters of the invention can provide that differentiation by providing confirmation of activity sensor localization in the target tissue and by offering levels of off-target immune system activity for comparative analysis of tumor-specific immune system activity.

Controls may include general target-specific reporters (e.g., responsive to enzymes differentially expressed in a target tissue regardless of disease status). The presence of such reporters in a sample provides confirmation that the activity sensors reached the target tissue and that tissue-status reporter levels (e.g., cancer progression related or immunological enzyme reporter levels) can be attributed to the target tissue. For example, in an I-O analysis application for lung cancer, immunological enzyme-sensitive activity sensors can be co-administered with lung-specific enzyme-sensitive activity sensors. Presence of lung-specific reporter in a patient sample is indicative that the activity sensors reached the target tissue and that immunological enzyme-sensitive reporter levels found in the sample are likely attributed to an anti-tumor response as opposed to an off-target immune response. On the other hand, presence of immunological enzyme-sensitive reporter in a patient sample without corresponding lung-specific reporters may indicate a false positive result caused by off-target immune system response.

Control reporters may also prevent false negative results by providing a baseline signal indicative of a successful assay. In the above example, the absence of both general lung-specific reporters and immunological enzyme-sensitive reporters in a patient sample may indicate that the assay has failed and that no clinical conclusions should be drawn regarding a lack of anti-tumor response. In certain embodiments, control reporters can be staged to be cleaved by enzymes along various stages of the administration route such that subsequent analysis can help troubleshoot the assay wherein the trail of reporters present in a patient sample indicates where along the administration route a problem may be occurring.

Levels of control reporters can also function as normalizers. The primary purpose of normalization is to remove sample-to-sample variability by correcting data for factors other than the reporter target. Normalization can be accomplished by dividing the target reporter level by a second control value. Immunological enzyme or cancer-specific enzyme reporter levels can be divided by any of the above control reporter levels to normalize the I-O response data. For example, as discussed below, immunological enzyme-sensitive activity sensors can be targeted to tumor tissue to provide immunological response information specific to the tumor tissue. In certain embodiments, non-targeted immunological enzyme-sensitive activity sensors can be co-administered to provide a comparative level of general immune system activity. The target-specific levels can be divided by the non-target-specific levels to normalize for general immune response and provide a more accurate picture of anti-tumor immune response.

In certain embodiments, test-enzyme-sensitive reporter levels (e.g., reporting immunological enzyme or other condition-indicative enzyme activity) are normalized against levels of control reporters sensitive to ubiquitous or target-specific enzymes that are not disease related. Such control levels are indicative of general assay function and normalization using them can help smooth sample-to-sample variation which can be especially useful in I-O therapy monitoring techniques that rely on sample-to-sample comparison of reporter measurements. As described herein, activity sensors act as synthetic biomarkers that can be programmed to provide non-invasive reporting of any enzyme level in a specific target tissue through engineering of an enzyme-specific cleavage site in the activity sensor. In certain embodiments, normalizer reporters and experimental reporters can be included on separate carriers. In preferred embodiments, activity sensors can include multiple cleavable reporter molecules that may be cleaved by the same or different enzymes. Accordingly, control or normalizer reporters can be included on a single carrier with the experimental reporter (e.g., an immunological enzyme-sensitive reporter). For example, the activity sensors may be a multi-arm polyethylene glycol (PEG) scaffold linked to four or more polypeptide reporters as the cleavable analytes. The cleavable linkers are specific for different enzymes whose activity is characteristic of a condition to be monitored (e.g., a certain stage or progression in cancerous tissue or an immune response). When administered to a patient, the activity sensors locate to a target tissue, where they are cleaved by the enzymes to release the detectable analytes. The analytes are detected in a patient sample such as urine. The detected analytes serve as a report of which enzymes are active in the tissue and, therefore, the associated condition or activity.

In various embodiments, experimental activity sensors can include tumor-localized activity sensors with cleavable reporters sensitive to immunological enzymes useful for prediction of I-O therapeutic response and detailed monitoring of cancer progression and evolution. Contextualized by control and normalizer data, I-O reporter levels can help predict therapeutic response, stage disease progression, and monitor therapeutic efficacy. Experimental activity sensors may include cleavable linkers sensitive to proteases differentially expressed in immune responses including inflammation and apoptosis. Activity sensors sensitive to proteases associated with necrotic cell death associated with natural tumor progression can also be included to provide additional information on cancer progression. Comparison of inflammation/apoptosis-related protease levels to necrosis-related protease levels can provide a more detailed view of cancer progression and I-O treatment response.

Caspases (cysteine-aspartic proteases, cysteine aspartases or cysteine-dependent aspartate-directed proteases) are associated with programmed cell death (e.g., apoptosis) and inflammation and, therefore, activity sensors engineered with caspase-cleavable reporters as described herein can provide a synthetic biomarker indicative of immune response. Other proteases indicative of an immune response include serine proteases such as granzymes, neutrophil elastase, cathepsin G, proteinase 3, chymase, and tryptase. As discussed below, those synthetic biomarkers can include tuning domains to localize the activity sensors to tumors in order to provide a tumor-specific picture of immune response useful in differentiating I-O therapeutic efficacy from systemic or off-target immune response.

Activity sensors can include a molecular carrier structure linked to one or more detectable analytes via cleavable linkers. The presence and amount of immunological enzymes as measured by activity sensor reporter levels in a patient sample and contextualized by control and/or normalizer data can be used to determine innate, artificial, or augmented immuno-oncological responses in a patient. For example, a baseline signal of caspase and serine protease reporters from tumor-localized activity sensors as verified by comparison/normalization to non-targeted immunological enzyme-sensitive activity sensor controls can indicate a non-responsive tumor when measured after treatment or an immunologically-cold tumor when measured before treatment. An indication of an immunologically-cold tumor can indicate that the patient is not likely to respond to checkpoint inhibitors. Slightly elevated signals of caspase and serine protease reporters from tumor-localized activity sensors measured pre-treatment can be indicative of an immunologically-hot tumor where the patient may be a good candidate for checkpoint inhibitors. A high signal of caspase and serine protease reporters from tumor-localized activity sensors measured during or after treatment may be indicative of a desired immuno-oncological response. In any of the above cases, determination of base-line, slightly elevated, or highly elevated levels of target-specific immunological enzymes can include comparison against levels of a control reporter such as a non-targeted immunological enzyme.

Reported levels of necrosis-related proteases such as calpain and cathepsin can provide information regarding necrotic cell death to supplement the immuno-oncological information and help differentiate between tumor progression and pseudoprogression. Control levels (e.g., non-targeted necrosis-related proteases) can help provide clear picture of tumor-specific necrosis.

The information provided using activity sensors of the invention can be used to distinguish hot tumors from cold tumors. Immunologically cold tumors refer to those tumors with few infiltrating T cells that do not provoke a strong response by the immune system. Hot tumors, in contrast, contain high levels of infiltrating T cells and more antigens and are more likely to trigger a strong immune response. Accordingly, a hot tumor, already recognized by the patient's immune system as a target, may be a good candidate for passive treatments such as a checkpoint inhibitor to simply augment the patient's existing immune response. Comparison of tumor-specific immune response to control values indicative of general immune system activity can assist in identifying immunologically hot tumors.

In certain embodiments, I-O activity sensors, acting as synthetic biomarkers, nucleic acids, proteins, dyes and the like, are administered and measured periodically to provide a chronological mapping of various enzyme levels. In addition to point-in-time information, the rate of change in measurements of the various enzyme levels can be examined to provide velocity information. Such a panel is useful for providing an indication of health, which is applicable even to healthy individuals and provides another data point beyond traditional longitudinal monitoring for disease progression and therapeutic response. In such velocity analyses, normalization across samples is important to dampen any effect of sample variability.

In various embodiments, activity sensors may take the form of cyclic peptides that are naturally resistant to off-target degradation. The target environment may be a tumor microenvironment in which a specific enzyme or set of enzymes are differentially-expressed. A cyclic peptide may be engineered with cleavage sites specific to enzymes in the tumor (e.g., unique enzymes expressed preferentially in the tumor) or to control enzymes for use in normalization. The engineered peptide, in its cyclic form, can travel through the blood and other potentially harsh environments protected against degradation by common non-specific proteases and without interacting in a meaningful way with off-target tissues. Only upon arrival within the specific target tissue and exposure to the required enzyme or combination of enzymes, the cyclic peptide is cleaved to produce a linear molecule that is capable of clearance and sample observation. For purposes of the application and as will be apparent upon consideration of the detailed description thereof, a linear peptide is any peptide that is not cyclic. Thus, for example, a linearized peptide may have various branch chains.

Cyclic peptides can be engineered with other cleavable linkages, such as ester bonds in the form of cyclic depsipeptides in which the degradation of the ester bond releases a linearized peptide ready to react with its target environment. Thioesters and other tunable bonds can be included in the cyclic peptide to create a timed-release in plasma or other environments. See Lin and Anseth, 2013 Biomaterials Science (Third Edition), pages 716-728, incorporated herein by reference.

Macrocyclic peptides may contain two or more protease-specific cleavage sequences and can require two or more protease-dependent hydrolytic events to release a reporter peptide or a bioactive compound. The protease-specific sequences can be different in various embodiments. In cases where cleavage of multiple sites is required to release the linearized peptide, different protease-specific sequences can increase specificity for the release as the presence of at least two different target-specific enzymes will be required. The specific and non-specific proteolysis susceptibility and rate can be tuned through manipulation of peptide sequence content, length, and cyclization chemistry.

Activity sensors may include additional molecular structures to influence trafficking of the peptides within the body, or timing of the enzymatic cleavage or other metabolic degradation of the particles. For use as controls and in normalization, it may be advantageous to target experimental activity sensors to one location while targeting control activity sensors to another. That task may be performed by molecular structures functioning as tuning domains, additional molecular subunits or linkers that are acted upon by the body to locate the activity sensor to the target tissue under controlled timing. For example, the tuning domain may target the particle to specific tissue or cell types. Trafficking may be influenced by the addition of molecular structures in the carrier polymer by, for example, increasing the size of a PEG scaffold to slow degradation in the body.

### BRIEF DESCRIPTION OF THE DRAWINGS

FIG. 1 diagrams steps of a method for monitoring cancer progression.

FIG. 2 shows an activity sensor.

FIG. 3 shows an engineered macrocyclic peptide.

### DETAILED DESCRIPTION

The invention provides detailed information on cancer-related immune responses through the use of localized activity sensors to report on differential expression of immunologically associated enzymes and the use of control or normalizing reporters to contextualize that data. Such activity sensors can include a variety of reporter molecules that are detectable in a body fluid sample such as urine but are only released from the body upon contact with cleavage enzymes associated with localized immune responses, cancer progression, or the control indicator. Accordingly, detection of the experimental reporters in the sample is indicative of the differential expression of the enzymes in the target tissue and the presence of the associated immune activity (e.g., immuno-therapeutic response or an immunologically-hot tumor). Detection and levels of the control reporters in the patient sample can be used to validate results, troubleshoot assay issues, and to normalize data across samples or patients. By preferentially targeting cancerous tissues and engineering cleavage sites specific to enzymes differentially expressed under various conditions, activity sensors of the invention can provide insight into cancer progression and predicted or actual immuno-therapeutic responses not possible with existing imaging or systemic monitoring techniques and can contextualize that data with control levels indicative of assay functionality and background or off-target activity.

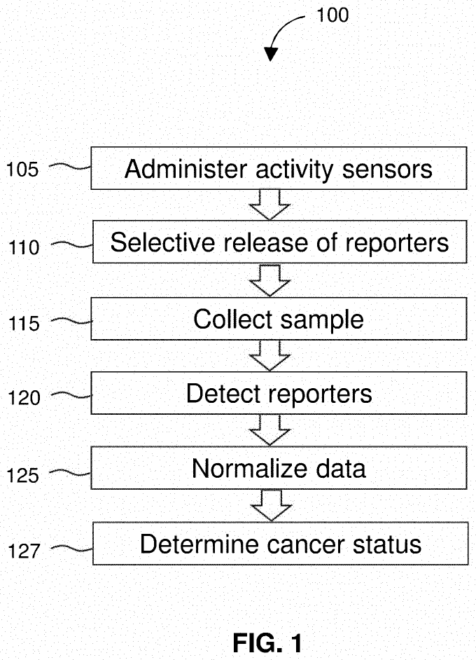

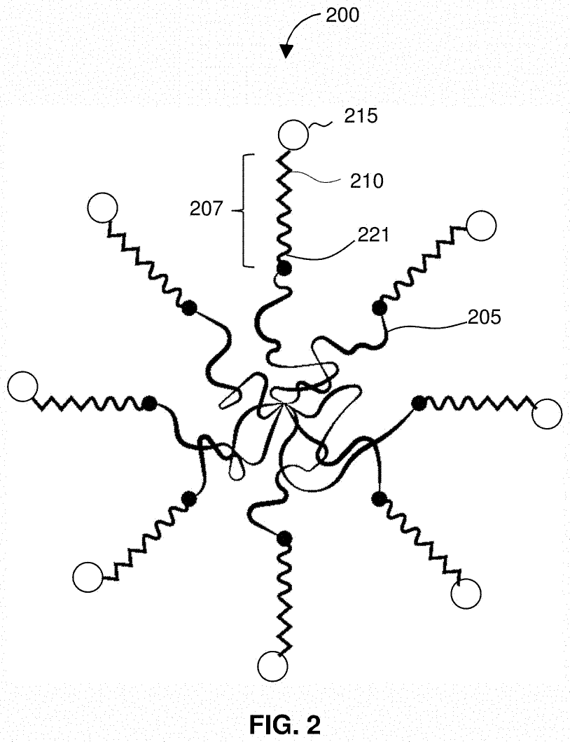

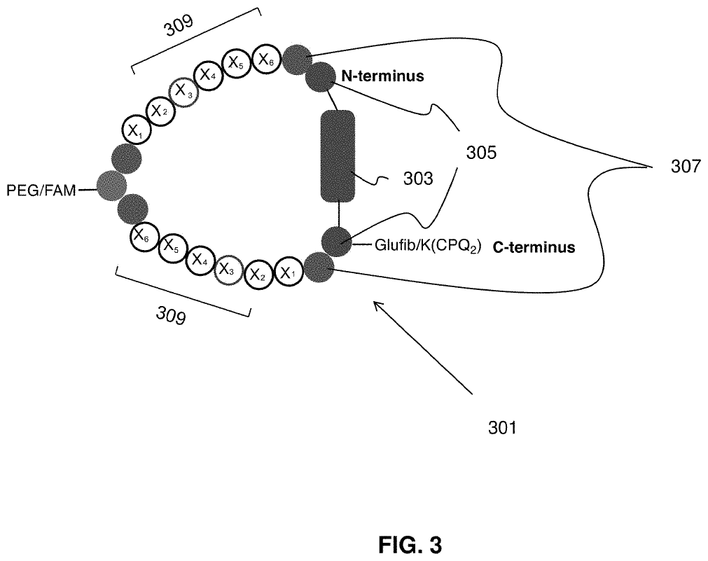

FIG. 1 diagrams steps of a method 100 for monitoring cancer progression. At step 105, activity sensors are administered to a patient. The patient may be suspected of having cancer, known to have cancer (active or in remission), at risk of developing cancer, and/or undergoing treatment for cancer including immuno-oncological (I-O) therapies. The activity sensors include reporters linked by cleavable linkers to a carrier (e.g., as shown in FIGS. 2 and 3). Experimental cleavable linkers are sensitive to an enzyme for which the level is indicative of a characteristic in the tumor environment (e.g., enzymes upregulated in expanding tumors or tumors in regression, or enzymes indicative of active or inhibited immune responses). Control cleavable linkers are also included. They can be on the same carrier as experimental cleavable linkers or on separate carriers. As discussed herein, depending on the enzyme activity the activity sensors are engineered to report on and the patient's disease and treatment status, information garnered from reporter levels in patient samples can be used to diagnose and/or stage the disease, monitor progression, predict responsiveness to a given therapy, and monitor therapeutic effectiveness including differentiating between anti-tumor immune response, general immune response, and tumor progression. Control reporters are used to contextualize or normalize the data obtained using from experimental reporters. Activity sensors can be administered by any suitable method. In preferred embodiments, the activity sensor is delivered intravenously or aerosolized and delivered to the lungs, for example, via a nebulizer. In other examples, the activity sensor may be administered to a subject transdermally, intradermally, intraarterially, intralesionally, intratumorally, intracranially, intraarticularly, intratumorally, intramuscularly, subcutaneously, orally, topically, locally, inhalation, injection, infusion, or by other method or any combination known in the art (see, for example, Remington's Pharmaceutical Sciences (1990), incorporated by reference).

At step 110, after administration of the activity sensors and localization of the activity sensor in the target tissue, the reporter is selectively released upon cleavage of the linker in the presence of the characteristic-indicative enzyme. Localization can be accomplished through the use of tuning domains including moieties preferentially concentrated in the target tissue (e.g., a specific tissue suspected of harboring cancer cells or concentrated in tumors generally). Upon release of the reporter, it can be cleared by the body into a fluid capable of non-invasive collection such as urine after transport to the blood stream and renal clearance. In various embodiments, control reporters may be cleaved by enzymes differentially expressed in a target tissue regardless of disease status. The presence of such reporters in a sample provides confirmation that the activity sensors reached the target tissue and that tissue-status reporter levels (e.g., cancer progression related or immunological enzyme reporter levels) can be attributed to the target tissue. In certain embodiments, control reporters may be present on non-targeted activity sensors, i.e., sensors that do not localize to the target tissue. Such control reporters can be cleavable by the same enzymes as the experimental reporters to provide an off-target enzyme level for normalization of the target-specific reporter information. For example, immunological enzyme or cancer-specific enzyme reporter levels can be divided by any of the above control reporter levels to normalize the I-O response data. For example, immunological enzyme-sensitive activity sensors can be targeted to tumor tissue to provide immunological response information specific to the tumor tissue. In certain embodiments, non-targeted immunological enzyme-sensitive activity sensors can be co-administered at step 105 to provide a comparative level of general immune system activity at step 120. The target-specific levels can be divided by the non-target-specific levels to normalize for general immune response and provide a more accurate picture of anti-tumor immune response.

At step 115, the sample, such as a urine sample, can be collected for analysis. At step 120, the sample can be analyzed and the presence and/or levels of the reporters in the sample can be detected.

At step 125, the experimental reporter levels can be normalized using control reporter levels. In certain embodiments, levels of control reporters can also function as normalizers. Normalization can remove sample-to-sample variability by dividing the target reporter levels by a control reporter value. Accordingly, any assay variability that may occur during steps 105-120 in different assays can be filtered out to provide a more accurate tracking of experimental enzyme levels across assays. Normalization can be used to control against variation from patient to patient or from assays of a single patient performed at different times.

An immunological enzyme may be an enzyme produced as part of an immune response. For example, an immunological enzyme may include an enzyme produced by immune cells.

In certain embodiments, test-enzyme-sensitive reporter levels (e.g., reporting immunological enzyme or other condition-indicative enzyme activity) can be normalized against levels of control reporters sensitive to ubiquitous or target-specific enzymes that are not disease related. Such control levels are indicative of general assay function and normalization using them can help smooth sample-to-sample variation which can be especially useful in I-O therapy monitoring techniques that rely on sample-to-sample comparison of reporter measurements.

At step 127, the enzyme levels indicated by the presence of the reporter can be used to determine a characteristic associated with the observed differential expression. As noted above, depending on the enzyme sensitivity engineered into the activity sensors used, reporter levels can be used to monitor disease progression and I-O therapy response or to predict responsiveness to various treatments (e.g., determine hot or cold status of a tumor). Levels of control reporters can be used in the analysis to determine the characteristic. For example an I-O analysis application for lung cancer, immunological enzyme-sensitive activity sensors may be co-administered with lung-specific enzyme-sensitive activity sensors at step 105. Detection of the lung-specific control reporters in the patient sample at step 120 is then indicative that the activity sensors reached the target tissue and that immunological enzyme-sensitive reporter levels found in the sample are likely attributed to an anti-tumor response as opposed to an off-target immune response. On the other hand, presence of immunological enzyme-sensitive reporter in a patient sample without corresponding lung-specific reporters may indicate a false positive result caused by off-target immune system response. Control reporters may also prevent false negative results by providing a baseline signal indicative of a successful assay. At step 120, the failure to detect target-specific or untargeted control reporters in the patient sample may indicate that the assay has failed and that no clinical conclusions should be drawn or that the process should be repeated.

In certain embodiments, control reporters can be staged to be cleaved by enzymes along various stages of the administration route such that subsequent analysis can help troubleshoot the assay wherein the trail of reporters present in a patient sample indicates where along the administration route a problem may be occurring. For example an ingestible lung-targeted activity sensor may include cleavable reporters sensitive to enzymes specific to the GI tract, the liver, blood, and lung tissue. Presence of GI tract and liver reporters alone may indicate a problem in the transfer of reporters from the liver to the bloodstream which can aid in troubleshooting the assay. In certain embodiments, an activity sensor may include reporters cleavable by off-target tissue-specific enzymes for tissues that are not part of the intended administration pathway and can provide information regarding off-target uptake.

Several proteases are known to be associated with inflammation and programmed cell death (e.g., including apoptosis, pyroptosis and necroptosis). The localized levels of those proteases are accordingly indicative of immune system activity. Similarly, the off-target or systemic levels of those proteases, as indicated by control reporters, is indicative of general immune system activity and can be compared to tumor tissue activity to normalize that data. Caspases (cysteine-aspartic proteases, cysteine aspartases or cysteine-dependent aspartate-directed proteases) are a family of protease enzymes including a cysteine in their active site that nucleophilically cleaves a target protein only after an aspartic acid residue. Caspase-1, Caspase-4, Caspase-5 and Caspase-11 are associated with inflammation. Serine proteases also function in apoptosis and inflammation and their differential expression is therefore also indicative of an immune response. Immune cells express serine proteases such as granzymes, neutrophil elastase, cathepsin G, proteinase 3, chymase, and tryptase.

In various embodiments, it may be useful to differentiate between programmed cell death indicative of an immune response and necrosis naturally found during tumor progression. In contrast to programmed cell death, where caspases and serine proteases are the primary proteases, calpains and lysosomal proteases (e.g., cathepsins B and D) are the key proteases in necrosis. Accordingly, calpain and cathepsin levels indicated by activity sensor reporter measurements can provide information regarding necrotic cell death to supplement the immuno-oncological information.

Activity sensors and methods of the invention can be applied to I-O treatments to observe I-O drug responses in patients. For example, activity sensors with cleavage sites sensitive to caspases, serine proteases, calpains, and cathepsins can be administered during or after I-O treatment and reporter levels in patient samples can be used to monitor therapeutic response. A baseline signal of caspases or serine proteases in patient samples is indicative of a non-responsive tumor. Such a baseline level may be determined through, for example, the use of untargeted control activity sensors. The baseline level can also be determined experimentally through data collected from patient populations or pre-treatment data from the patient undergoing treatment. Increased signals of caspases and serine proteases during or after treatment relative to a baseline level can be indicative of a desired immuno-oncological response. Tracking the levels of calpain or cathepsin signals can provide additional information on non-immunological cell death that may be associated with tumor progression. Levels of control reporters can be used to normalize that data across samples to account for assay variability.

Activity sensors act as synthetic biomarkers that can be programmed to provide non-invasive reporting of any enzyme level in a specific target tissue through engineering of an enzyme-specific cleavage site in the activity sensor. When administered to a patient, the activity sensors locate to a target tissue using, for example, target-specific tuning domains. Once localized, they are cleaved by the enzymes to release the detectable analytes. The analytes are detected in a patient sample such as a urine sample. The detected analytes serve as a report of which enzymes are active in the tissue and, therefore, the associated condition or activity. Localization allows activity sensors to report on the conditions of a target tissue without contamination of off-target information. That ability is useful in differentiating anti-tumor immune responses indicative of successful I-O treatment from an off-target immune response that may, for example, be occurring in response to a viral infection. The ability to systemically distribute or localize is also useful for providing control data including systemic levels of the target reporter or target-specific levels of a control enzyme. For example a general increase in immunological enzymes (e.g., caspases or serine proteases) may result from a systemic or off-target immune response such as a viral infection. The ability of the invention to provide tumor-specific information regarding immune system activity along with control data regarding background immune activity avoids misinterpretation of a general immune response as a desired anti-tumor response.

Activity sensors and methods of the invention can also be used to evaluate patient suitability for an I-O therapy. For example, activity sensors can report on enzymes differentially expressed in a patient's natural immune recognition and response to cancerous tissue. Such activity sensors can be administered before any I-O treatment in order to differentiate between hot tumors and cold tumors. Where a patient has a tumor that contains high levels of infiltrating T cells and more antigens, they may be a good candidate for passive treatments such as a checkpoint inhibitor to augment the patient's existing immune response. Checkpoint proteins include CTLA-4 (cytotoxic T lymphocyte associated protein 4), PD-1 (programmed cell death protein 1), and PD-L1 (programmed death ligand 1) are known to mask tumors from immune detection or response and various inhibitors for each are known. Where activity sensors sensitive to immune system recognition indicate a hot tumor, such checkpoint inhibitor therapies may be indicated. Comparison to systemic immunological enzyme levels from control reporters can provide the necessary data for establishing a baseline level. A higher-than-baseline level of increased level of caspase or serine protease activity in a pre-treatment tumor can be observed using activity sensors as discussed herein and would indicate some immune system recognition and activity at the tumor site. The presence of an innate immune recognition and response supports a conclusion that cancer progression is reliant on checkpoint protein manipulation and the administration of a checkpoint inhibitor may prove effective for that patient.

Enzyme-specific reporters, including experimental and control reporters can be multiplexed on single activity sensors or in many different activity sensors that are administered and analyzed simultaneously. The reporter molecules can be specific for each enzyme such that they can be distinguished in multiplex analysis. In certain embodiments, I-O activity sensors, acting as synthetic biomarkers, may be administered and measured periodically to provide a chronological mapping of various enzyme levels. Studies have found that biomarker velocity (the rate of change in biomarker levels over time) may be a better indicator of disease progression (or regression) than any single threshold. The same principle can be applied to the activity sensors of the invention acting as synthetic biomarkers. The ability to normalize data from different assays using control data is particularly useful in such velocity analyses.

Activity sensors can include a carrier, at least one reporter linked to the carrier and at least one tuning domain that modifies a distribution or residence time of the activity sensor within a subject when administered to the subject. The activity sensor may be designed to detect and report enzymatic activity in the body, for example, enzymes that are differentially expressed during immune responses or during tumor progression or regression. Dysregulated proteases have important consequences in the progression of diseases such as cancer in that they may alter cell signaling, help drive cancer cell proliferation, invasion, angiogenesis, avoidance of apoptosis, and metastasis.

The activity sensor may be tuned via the tuning domains in numerous ways to facilitate detecting enzymatic activity within the body in specific cells or in a specific tissue. For example, the activity sensor may be tuned to promote distribution of the activity sensor to the specific tissue or to improve a residence time of the activity sensor in the subject or in the specific tissue. Tuning domains may include, for example, molecules localized in rapidly replicating cells to better target tumor tissue.

When administered to a subject, the activity sensor is trafficked through the body and may diffuse from the systemic circulation to a specific tissue, where the reporter may be cleaved via enzymes indicative of cancer progression or immune response. The detectable analyte may then diffuse back into circulation where it may pass renal filtration and be excreted into urine, whereby detection of the detectable analyte in the urine sample indicates enzymatic activity in the target tissue.

The carrier may be any suitable platform for trafficking the reporters through the body of a subject, when administered to the subject. The carrier may be any material or size suitable to serve as a carrier or platform. Preferably the carrier is biocompatible, non-toxic, and non-immunogenic and does not provoke an immune response in the body of the subject to which it is administered. The carrier may also function as a targeting means to target the activity sensor to a tissue, cell or molecule. In some embodiments the carrier domain is a particle such as a polymer scaffold. The carrier may, for example, result in passive targeting to tumors or other specific tissues by circulation. Other types of carriers include, for example, compounds that facilitate active targeting to tissue, cells or molecules. Examples of carriers include, but are not limited to, nanoparticles such as iron oxide or gold nanoparticles, aptamers, peptides, proteins, nucleic acids, polysaccharides, polymers, antibodies or antibody fragments and small molecules.

The carrier may include a variety of materials such as iron, ceramic, metallic, natural polymer materials such as hyaluronic acid, synthetic polymer materials such as poly-glycerol sebacate, and non-polymer materials, or combinations thereof. The carrier may be composed in whole or in part of polymers or non-polymer materials, such as alumina, calcium carbonate, calcium sulfate, calcium phosphosilicate, sodium phosphate, calcium aluminate, and silicates. Polymers include, but are not limited to: polyamides, polycarbonates, polyalkylenes, polyalkylene glycols, polyalkylene oxides, cellulose ethers, cellulose esters, nitro celluloses, polymers of acrylic and methacrylic esters, methyl cellulose, ethyl cellulose, and hydroxypropyl cellulose. Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, poly-anhydrides, polyurethanes, and natural polymers such as alginate and other polysaccharides including dextran and cellulose, collagen, albumin and other proteins, copolymers and mixtures thereof. In general, these biodegradable polymers degrade either by enzymatic hydrolysis or exposure to water in vivo, by surface or bulk erosion. These biodegradable polymers may be used alone, as physical mixtures (blends), or as co-polymers.

In preferred embodiments, the carrier includes biodegradable polymers so that whether the reporter is cleaved from the carrier, the carrier will be degraded in the body. By providing a biodegradable carrier, accumulation and any associated immune response or unintended effects of intact activity sensors remaining in the body may be minimized.

Other biocompatible polymers include PEG, PVA and PVP, which are all commercially available. PVP is a non ionogenic, hydrophilic polymer having a mean molecular weight ranging from approximately 10,000 to 700,000 and has the chemical formula (C6H9NO)[n]. PVP is also known as poly[1 (2 oxo 1 pyrrolidinyl)ethylene]. PVP is nontoxic, highly hygroscopic and readily dissolves in water or organic solvents.

Polyvinyl alcohol (PVA) is a polymer prepared from polyvinyl acetates by replacement of the acetate groups with hydroxyl groups and has the chemical formula (CH2CHOH)[n]. Most polyvinyl alcohols are soluble in water.

Polyethylene glycol (PEG), also known as poly(oxyethylene) glycol, is a condensation polymer of ethylene oxide and water. PEG refers to a compound that includes repeating ethylene glycol units. The structure of PEG may be expressed as H—(O—CH2-CH2)n—OH. PEG is a hydrophilic compound that is biologically inert (i.e., non-immunogenic) and generally considered safe for administration to humans.

When PEG is linked to a particle, it provides advantageous properties, such as improved solubility, increased circulating life, stability, protection from proteolytic degradation, reduced cellular uptake by macrophages, and a lack of immunogenicity and antigenicity. PEG is also highly flexible and provides bio-conjugation and surface treatment of a particle without steric hindrance. PEG may be used for chemical modification of biologically active compounds, such as peptides, proteins, antibody fragments, aptamers, enzymes, and small molecules to tailor molecular properties of the compounds to particular applications. Moreover, PEG molecules may be functionalized by the chemical addition of various functional groups to the ends of the PEG molecule, for example, amine-reactive PEG (BS (PEG)n) or sulfhydryl-reactive PEG (BM (PEG)n).

In certain embodiments, the carrier is a biocompatible scaffold, such as a scaffold including polyethylene glycol (PEG). In a preferred embodiment, the carrier is a biocompatible scaffold that includes multiple subunits of covalently linked polyethylene glycol maleimide (PEG-MAL), for example, an 8-arm PEG-MAL scaffold. A PEG-containing scaffold may be selected because it is biocompatible, inexpensive, easily obtained commercially, has minimal uptake by the reticuloendothelial system (RES), and exhibits many advantageous behaviors. For example, PEG scaffolds inhibit cellular uptake of particles by numerous cell types, such as macrophages, which facilitates proper distribution to a specific tissues and increases residence time in the tissue.

An 8-arm PEG-MAL is a type of multi-arm PEG derivative that has maleimide groups at each terminal end of its eight arms, which are connected to a hexaglycerol core. The maleimide group selectively reacts with free thiol, SH, sulfhydryl, or mercapto group via Michael addition to form a stable carbon sulfur bond. Each arm of the 8-arm PEG-MAL scaffold may be conjugated to peptides, for example, via maleimide-thiol coupling or amide bonds.

The PEG-MAL scaffold may be of various sizes, for example, a 10 kDa scaffold, a 20 kDa scaffold, a 40 kDa scaffold, or a greater than 40 kDa scaffold. The hydrodynamic diameter of the PEG scaffold in phosphate buffered saline (PBS) may be determined by various methods known in the art, for example, by dynamic light scattering. Using such techniques, the hydrodynamic diameter of a 40 kDa PEG-MAL scaffold was measured to be approximately 8 nm. In preferred embodiments, a 40 kDa PEG-MAL scaffold is provided as the carrier when the activity sensor is administered subcutaneously because the activity sensor readily diffuses into systemic circulation but is not readily cleared by the reticuloendothelial system.

The size of the PEG-MAL scaffold affects the distribution and residence time of the activity sensor in the body because particles smaller than about 5 nm in diameter are efficiently cleared through renal filtration of the body, even without proteolytic cleavage. Further, particles larger than about 10 nm in diameter often drain into lymphatic vessels. In one example, where a 40 kDa 8-arm PEG-MAL scaffold was administered intravenously, the scaffold was not renally cleared into urine.

The reporter may be any reporter susceptible to an enzymatic activity, such that cleavage of the reporter indicates that enzymatic activity. The reporter is dependent on enzymes that are active in a specific disease state. For example, tumors are associated with a specific set of enzymes. For a tumor, the activity sensor may be designed with an enzyme susceptible site that matches that of the enzymes expressed by the tumor or other diseased tissue. Alternatively, the enzyme-specific site may be associated with enzymes that are ordinarily present but are absent in a particular disease state. In this example, a disease state would be associated with a lack of signal associated with the enzyme, or reduced levels of signal compared to a normal reference (e.g., from a control reporter) or prior measurement in a healthy subject.

In various embodiments, the reporter includes a naturally occurring molecule such as a peptide, nucleic acid, a small molecule, a volatile organic compound, an elemental mass tag, or a neoantigen. In other embodiments, the reporter includes a non-naturally occurring molecule such as D-amino acids, synthetic elements, or synthetic compounds. The reporter may be a mass-encoded reporter, for example, a reporter with a known and individually-identifiable mass, such as a polypeptide with a known mass or an isotope.

An enzyme may be any of the various proteins produced in living cells that accelerate or catalyze the metabolic processes of an organism. Enzymes act on substrates. The substrate binds to the enzyme at a location called the active site before the reaction catalyzed by the enzyme takes place. Generally, enzymes include but are not limited to proteases, glycosidases, lipases, heparinases, and phosphatases. Examples of enzymes that are associated with disease in a subject include but are not limited to MMP, MMP-2, MMP-7, MMP-9, kallikreins, cathepsins, seprase, glucose-6-phosphate dehydrogenase (G6PD), glucocerebrosidase, pyruvate kinase, tissue plasminogen activator (tPA), a disintegrin and metalloproteinase (ADAM), ADAMS, ADAM15, and matriptase. The detected enzymatic activity may be activity of any type of enzyme, for example, proteases, kinases, esterases, peptidases, amidases, oxidoreductases, transferases, hydrolases, lysases, isomerases, or ligases.

Examples of substrates for disease-associated enzymes include but are not limited to Interleukin 1 beta, IGFBP-3, TGF-beta, TNF, FASL, HB-EGF, FGFR1, Decorin, VEGF, EGF, IL2, IL6, PDGF, fibroblast growth factor (FGF), and tissue inhibitors of MMPs (TIMPs). Enzymes indicative of immune response can include, for example, tissue remodeling enzymes.

The tuning domains may include any suitable material that modifies a distribution or residence time of the activity sensor within a subject when the activity sensor is administered to the subject. For example, the tuning domains may include PEG, PVA, or PVP. In another example, the tuning domains may include a polypeptide, a peptide, a nucleic acid, a polysaccharide, volatile organic compound, hydrophobic chains, or a small molecule.

FIG. 2 shows an activity sensor 200 with carrier 205, reporters 207, and tuning domains 215. As illustrated, carrier 205 is a biocompatible scaffold that includes multiple subunits of covalently linked polyethylene glycol maleimide (PEG-MAL). Carrier 205 is an 8-arm PEG-MAL scaffold with a molecular weight between about 20 and 80 kDa. Reporter 207 is a polypeptide including a region susceptible to an identified protease. Activity of the identified protease to cleave the reporter indicates the disease. Reporter 207 includes a cleavable substrate 221 connected to detectable analyte 210. When a cleavage by the identified protease occurs upon cleavable substrate 221, detectable analyte 210 is released from activity sensor 200 and may pass out of the tissue, excreted from the body and detected.

In various embodiments, activity sensors may include cyclic peptides that are structurally resistant to non-specific proteolysis and degradation in the body. Cyclic peptides can include protease-specific substrates or pH-sensitive bonds that allow the otherwise non-reactive cyclic peptide to release a reactive reporter molecule in response to the presence of the enzymes discussed herein. Cyclic peptides can require cleavage at a plurality of cleavage sites to increase specificity. The plurality of sites can be specific for the same or different proteases. Polycyclic peptides can be used comprising 2, 3, 4, or more cyclic peptide structures with various combinations of enzymes or environmental conditions required to linearize or release the functional peptide or other molecule. Cyclic peptides can include depsipeptides wherein hydrolysis of one or more ester bonds releases the linearized peptide. Such embodiments can be used to tune the timing of peptide release in environments such as plasma.

FIG. 3 shows an exemplary cyclic peptide 301 having a protease-specific substrate 309 and a stable cyclization linker 303. The protease-specific substrate 309 may comprise any number of amino acids in any order. For example, X₁ may be glycine. X₂ may be serine. X₃ may be aspartic acid. X₄ may be phenylalanine. X₅ may be glutamic acid. X₆ may be isoleucine. The N-terminus and C-terminus, coupled to the cyclization linker 303 comprise cyclization residues 305. The peptide may be engineered to address considerations such as protease stability, steric hindrance around cleavage site, macrocycle structure, and rigidity/flexibility of peptide chain. The type and number of spacer residues 307 can be chosen to address and alter many of those properties by changing the spacing between the various functional sites of the cyclic peptide. The cyclization linker and the positioning and choice of cyclization residues can also impact the considerations discussed above. Tuning domains such as PEG and/or reporters such as FAM can be included in the cyclic peptide.

The biological sample may be any sample from a subject in which the reporter may be detected. For example, the sample may be a tissue sample (such as a blood sample, a hard tissue sample, a soft tissue sample, etc.), a urine sample, saliva sample, mucus sample, fecal sample, seminal fluid sample, or cerebrospinal fluid sample.

### Reporter Detection

Reporter molecules, released from activity sensors of the invention, may be detected by any suitable detection method able to detect the presence of quantity of molecules within the detectable analyte, directly or indirectly. For example, reporters may be detected via a ligand binding assay, which is a test that involves binding of the capture ligand to an affinity agent. Reporters may be directly detected, following capture, through optical density, radioactive emissions, or non-radiative energy transfers. Alternatively, reporters may be indirectly detected with antibody conjugates, affinity columns, streptavidin-biotin conjugates, PCR analysis, DNA microarray, or fluorescence analysis.

A ligand binding assay often involves a detection step, such as an ELISA, including fluorescent, colorimetric, bioluminescent and chemiluminescent ELISAs, a paper test strip or lateral flow assay, or a bead-based fluorescent assay.

In one example, a paper-based ELISA test may be used to detect the liberated reporter in urine. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Urine from the urine sample taken from the subject may then be added to the test spots, then streptavidin alkaline phosphate (ALP) conjugate may be added to the test spots, as the detection antibody. Bound ALP may then be exposed to a color reacting agent, such as BCIP/NBT (5-bromo-4-chloro-3′-indolyphosphate p-toluidine salt/nitro-blue tetrazolium chloride), which causes a purple colored precipitate, indicating presence of the reporter.

In another example, volatile organic compounds may be detected by analysis platforms such as gas chromatography instrument, a breathalyzer, a mass spectrometer, or use of optical or acoustic sensors.

Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

A modified breathalyzer instrument may also be used to detect volatile organic compounds. In a traditional breathalyzer that is used to detect an alcohol level in blood, a subject exhales into the instrument, and any ethanol present in the subject's breath is oxidized to acetic acid at the anode. At the cathode, atmospheric oxygen is reduced. The overall reaction is the oxidation of ethanol to acetic acid and water, which produces an electric current that may be detected and quantified by a microcontroller. A modified breathalyzer instrument exploiting other reactions may be used to detect various volatile organic compounds.

Mass spectrometry may be used to detect and distinguish reporters based on differences in mass. In mass spectrometry, a sample is ionized, for example by bombarding it with electrons. The sample may be solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis may be performed directly on the biological sample or the detectable analyte may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis.

Detecting the detectable analyte may provide a qualitative assessment (e.g., whether the detectable analyte is present or absent) or a quantitative assessment (e.g., the amount of the detectable analyte present) to indicate a comparative activity level of the enzymes. The quantitative value may be calculated by any means, such as, by determining the percent relative amount of each fraction present in the sample. Methods for making these types of calculations are known in the art.

The detectable analyte may be labeled. For example, a label may be added directly to a nucleic acid when the isolated detectable analyte is subjected to PCR. For example, a PCR reaction performed using labeled primers or labeled nucleotides will produce a labeled product. Labeled nucleotides, such as fluorescein-labeled CTP are commercially available. Methods for attaching labels to nucleic acids are well known to those of ordinary skill in the art and, in addition to the PCR method, include, for example, nick translation and end-labeling.

Labels suitable for use in the reporter include any type of label detectable by standard methods, including spectroscopic, photochemical, biochemical, electrical, optical, or chemical methods. The label may be a fluorescent label. A fluorescent label is a compound including at least one fluorophore. Commercially available fluorescent labels include, for example, fluorescein phosphoramidites, rhodamine, polymethadine dye derivative, phosphores, Texas red, green fluorescent protein, CY3, and CY5.

Other known techniques, such as chemiluminescence or colormetrics (enzymatic color reaction), can also be used to detect the reporter. Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET), such as fluorescence resonance energy transfer (FRET). Cleavage of the peptide results in separation of the chromophore and fluorophore, removal of the quench, and generation of a subsequent signal measured from the donor fluorophore. Examples of FRET pairs include 5-Carboxyfluorescein (5-FAM) and CPQ2, FAM and DABCYL, Cy5 and QSY21, Cy3 and QSY7.

In various embodiments, the activity sensor may include ligands to aid it targeting particular tissues or organs. When administered to a subject, the activity sensor is trafficked in the body through various pathways depending on how it enters the body. For example, if activity sensor is administered intravenously, it will enter systemic circulation from the point of injection and may be passively trafficked through the body.

For the activity sensor to respond to enzymatic activity within a specific cell, at some point during its residence time in the body, the activity sensor must come into the presence of the enzyme and have an opportunity to be cleaved and linearized by the enzyme to release the linearized reporter or therapeutic molecule. From a targeting perspective, it is advantageous to provide the activity sensor with a means to target specific cells or a specific tissue type where such enzymes of interest may be present. To achieve this, ligands for receptors of the specific cell or specific tissue type may be provided as the tuning domains and linked to polypeptide.

Cell surface receptors are membrane-anchored proteins that bind ligands on the outside surface of the cell. In one example, the ligand may bind ligand-gated ion channels, which are ion channels that open in response to the binding of a ligand. The ligand-gated ion channel spans the cell's membrane and has a hydrophilic channel in the middle. In response to a ligand binding to the extracellular region of the channel, the protein's structure changes in such a way that certain particles or ions may pass through. By providing the activity sensor with tuning domains that include ligands for proteins present on the cell surface, the activity sensor has a greater opportunity to reach and enter specific cells to detect enzymatic activity within those cells.

By providing the activity sensor with tuning domains, distribution of the activity sensor may be modified because ligands may target the activity sensor to specific cells or specific tissues in a subject via binding of the ligand to cell surface proteins on the targeted cells. The ligands of tuning domains may be selected from a group including a small molecule; a peptide; an antibody; a fragment of an antibody; a nucleic acid; and an aptamer.

Once activity sensor reaches the specific tissue, ligands may also promote accumulation of the activity sensor in the specific tissue type. Accumulating the activity sensor in the specific tissue increases the residence time of the activity sensor and provides a greater opportunity for the activity sensor to be enzymatically cleaved by proteases in the tissue, if such proteases are present.

When the activity sensor is administered to a subject, it may be recognized as a foreign substance by the immune system and subjected to immune clearance, thereby never reaching the specific cells or specific tissue where the specific enzymatic activity can release the therapeutic compound or reporter molecule. Furthermore, generation of an immune response can defeat the purpose of immune-response-sensitive activity sensors. To inhibit immune detection, it is preferable to use a biocompatible carrier so that it does not elicit an immune response, for example, a biocompatible carrier may include one or more subunits of polyethylene glycol maleimide. Further, the molecular weight of the polyethylene glycol maleimide carrier may be modified to facilitate trafficking within the body and to prevent clearance of the activity sensor by the reticuloendothelial system. Through such modifications, the distribution and residence time of the activity sensor in the body or in specific tissues may be improved.

In various embodiments, the activity sensor may be engineered to promote diffusion across a cell membrane. As discussed above, cellular uptake of activity sensors has been well documented. See Gang. Hydrophobic chains may also be provided as tuning domains to facilitate diffusion of the activity sensor across a cell membrane may be linked to the activity sensor.

The tuning domains may include any suitable hydrophobic chains that facilitate diffusion, for example, fatty acid chains including neutral, saturated, (poly/mono) unsaturated fats and oils (monoglycerides, diglycerides, triglycerides), phospholipids, sterols (steroid alcohols), zoosterols (cholesterol), waxes, and fat-soluble vitamins (vitamins A, D, E, and K).

In some embodiments, the tuning domains include cell-penetrating peptides. Cell-penetrating peptides (CPPs) are short peptides that facilitate cellular intake/uptake of activity sensors of the disclosure. CPPs preferably have an amino acid composition that either contains a high relative abundance of positively charged amino acids such as lysine or arginine or has sequences that contain an alternating pattern of polar/charged amino acids and non-polar, hydrophobic amino acids. See Milletti, 2012, Cell-penetrating peptides: classes, origin, and current landscape, Drug Discov Today 17:850-860, incorporated by reference. Suitable CPPs include those known in the literature as Tat, R6, R8, R9, Penetratin, pVEc, RRL helix, Shuffle, and Penetramax. See Kristensen, 2016, Cell-penetrating peptides as tools to enhance non-injectable delivery of biopharmaceuticals, Tissue Barriers 4(2):e1178369, incorporated by reference.

In certain embodiments, an activity sensor may include a biocompatible polymer as a tuning domain to shield the activity sensor from immune detection or inhibit cellular uptake of the activity sensor by macrophages.

When a foreign substance is recognized as an antigen, an antibody response may be triggered by the immune system. Generally, antibodies will then attach to the foreign substance, forming antigen-antibody complexes, which are then ingested by macrophages and other phagocytic cells to clear those foreign substances from the body. As such, when an activity sensor enters the body, it may be recognized as an antigen and subjected to immune clearance, preventing the activity sensor from reaching a specific tissue to detect enzymatic activity. To inhibit immune detection of the activity sensor, for example, PEG tuning domains may be linked to the activity sensor. PEG acts as a shield, inhibiting recognition of the activity sensor as a foreign substance by the immune system. By inhibiting immune detection, the tuning domains improve the residence time of the activity sensor in the body or in a specific tissue.

Enzymes have a high specificity for specific substrates by binding pockets with complementary shape, charge and hydrophilic/hydrophobic characteristic of the substrates. As such, enzymes can distinguish between very similar substrate molecules to be chemoselective (i.e., preferring an outcome of a chemical reaction over an alternative reaction), regioselective (i.e., preferring one direction of chemical bond making or breaking over all other possible directions), and stereospecific (i.e., only reacting on one or a subset of stereoisomers).

Steric effects are nonbonding interactions that influence the shape (i.e., conformation) and reactivity of ions and molecules, which results in steric hindrance. Steric hindrance is the slowing of chemical reactions due to steric bulk, affecting intermolecular reactions. Various groups of a molecule may be modified to control the steric hindrance among the groups, for example to control selectivity, such as for inhibiting undesired side-reactions. By providing the activity sensor with tuning domains such as spacer residues between the carrier and the cleavage site and/or any bioconjugation residue, steric hindrance among components of activity sensor may be minimized to increase accessibility of the cleavage site to specific proteases. Alternatively, steric hindrance can be used as described above to prevent access to the cleavage site until an unstable cyclization linker (e.g., an ester bond of a cyclic depsipeptide) has degraded. Such unstable cyclization linkers can be other known chemical moieties that hydrolyze in defined conditions (e.g., pH or presence of a certain analyte) which may be selected to respond to specific characteristics of a target environment.

In various embodiments, activity sensors may include D-amino acids aside from the target cleavage site to further prevent non-specific protease activity. Other non-natural amino acids may be incorporated into the peptides, including synthetic non-native amino acids, substituted amino acids, or one or more D-amino acids.

In some embodiments, tuning domains may include synthetic polymers such as polymers of lactic acid and glycolic acid, polyanhydrides, polyurethanes, and natural polymers such as alginate and other polysaccharides including dextran and cellulose, collagen, albumin and other hydrophilic proteins, zein and other prolamines and hydrophobic proteins, copolymers and mixtures thereof.

One of skill in the art would know what peptide segments to include as protease cleavage sites in an activity sensor of the disclosure. One can use an online tool or publication to identify cleavage sites. For example, cleavage sites are predicted in the online database PROSPER, described in Song, 2012, PROSPER: An integrated feature-based tool for predicting protease substrate cleavage sites, PLoSOne 7(11):e50300, incorporated by reference. Any of the compositions, structures, methods or activity sensors discussed herein may include, for example, any suitable cleavage site, as well as any further arbitrary polypeptide segment to obtain any desired molecular weight. To prevent off-target cleavage, one or any number of amino acids outside of the cleavage site may be in a mixture of the D and/or the L form in any quantity.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of monitoring cancer progression comprising:
administering to a patient suspected of having cancer an activity sensor comprising a carrier linked to a reporter molecule by a cleavable linker that is cleaved in the presence of a characteristic of a tumor;
administering to the patient a control activity sensor comprising a carrier linked to a control reporter molecule by a control cleavable linker that is cleaved by a control molecule;
collecting a sample from the patient;
analyzing the sample to detect the presence or lack of the reporter and the control reporter, wherein presence of the reporter is indicative of the characteristic.

2. The method of claim 1, wherein absence of the control reporter in the sample is indicative of a failed assay.

3. The method of claim 1, wherein said characteristic is an enzyme present in a tumor.

4. The method of claim 1, wherein said control molecule is an enzyme.

5. The method of claim 1, wherein the analyzing step further comprises quantifying a level of the reporter and a level of the control reporter in the sample, the method further comprising:
dividing the level of the reporter by the level of the control reporter to determine a normalized reporter level.

6. The method of claim 5, further comprising:
periodically repeating the administering, collecting, and analyzing steps to prepare a chronological series of normalized reporter levels, and
determining a velocity of the characteristic of the tumor environment

7. The method of claim 3, wherein the enzyme is an immunological enzyme.

8. The method of claim 7, wherein the patient is undergoing immuno-oncological treatment and the presence of the reporter is indicative of therapeutic effect of the immuno-oncological treatment.

9. The method of claim 8, wherein the activity sensor further comprises a tuning domain operable to localize the activity sensor in a target tumor.

10. The method of claim 7, wherein the patient has not undergone immuno-oncological treatment and the presence of the reporter is indicative of a predicted therapeutic response to a checkpoint inhibitor therapy.

11. The method of claim 1, further comprising stratifying the patient in a clinical trial based on the detection of the reporter in the sample.

12. The method of claim 7, wherein the immunological enzyme is selected from the group consisting of a caspase and a serine protease.

13. The method of claim 1, wherein the activity sensor comprises a tuning domain operable to localize the activity sensor in a target tumor.

14. The method of claim 13, wherein the control activity sensor comprises the tuning domain and the control enzyme is not an immunological enzyme but is differentially expressed in the target tumor, and wherein presence of the control reporter is indicative of target localization.

15. The method of claim 14, wherein the reporter molecule and the control reporter molecule are both linked to the same carrier.

16. The method of claim 13, wherein the control activity sensor does not comprise the targeting domain and the control enzyme is the immunological enzyme,
wherein presence of the control reporter is indicative of target localization, and
wherein the analyzing step further comprises quantifying a level of the reporter and a level of the control reporter in the sample, the method further comprising:
comparing the level of the reporter to the level of the control reporter to identify a tumor-specific immune response.
